(19) United States
(12) Patent Application Publication
(10) Pub. No.: US 2007/0059385 A1
(43) Pub. Date: Mar. 15, 2007

(54) Safe pharmaceutical composition for treating and preventing cancer and method of use

(76) Inventor: Yaguang Liu, Little Egg Harbor, NJ (US)

Correspondence Address: Yaguang Liu, 22 Sunrise Bay Blvd., Little Egg Harbor, NJ, 08087, US

(21) Appl. No.: 11/223,860

(22) Filed: Sep. 12, 2005

(51) Int. Cl. A61K 36/185; C12P 17/00; A61K 31/55

(52) U.S. Cl. 424/725; 514/214.03; 435/117

(57) ABSTRACT

The present invention provides a new method for extracting Homoharringtonine (HHT) by culture cells or culture plant tissues. Also, disclosed are methods of obtaining HHT from semi-synthesis and biosynthesis. The present invention disclosed that HHT combined with some botanical drugs could induce cancer cells to resemble normal cells. To add some botanical drugs combined with HHT can significantly increase anticancer effects of HHT. These drugs include Matrine (MAT), Guanzhongsu (GU), Maidongsu (MU), and Indirubin (IND). The experimental data showed that above drugs have strong synergisms effects for treating leukemia and other cancer cells and more safe.

### BACKGROUND OF THE INVENTION

The present invention provides a new method for extracting Homoharringtonine (HHT) by culture cells and plant tissues. Also, disclosed are methods of obtaining HHT from semi-synthesis and biosynthesis.

To add some botanical drugs combined with HHT can significantly increase anticancer effects of HHT. These drugs include Matrine (MAT), Guanzhongsu (GU), Maidongsu (MU), and Indirubin (IND). The experimental data showed that above drugs have strong synergisms effects for treating leukemia and other cancer cells and more safe.

### Description of the Prior Art

Cancer is the second leading cause of death in the United States, and the incidence of cancer continues to climb annually. In recent years, about 1 million new cases of cancer are diagnosed yearly in the U.S. About half million people and 7 million people of annual deaths are in the US and in the world, respectively. A lot of anticancer drugs including chemical and antibiotics have effects to kill cancer cells. But it also kills off some normal human cells, appears many kinds of side effects, among them the inhibition of bone marrow and tract are the most common.

Cyclopho-sphamide, for example, is a chemotherapeutic drug, which is highly effective against a wide range of human cancer. Cyclophosphamide established a role in the treatment of some major cancer types including Lymphomas, Acute Lymphatic leukemia, Chronic Lymphatic Leukemia, Breast, Pulmonal, Ovarial cancer and Tumor or marrow mulciple, Osseous, Sarcoma etc. Unfortunately, Cyclophosphamide has high toxicity, for example, it does damage to hemotopoietic organs, alimentary tract and decrease immune function. The toxicity of other anti-cancer medicines, for example, Fluorouracil, Mustine and 6-Mercaptopurine, etc. is higher than Cyclophosphamide. Some antibiotics are effective anticancer drugs, for example, Adriamycin is used for treatment of some cancers including leukemia, gastric pancreatic, breast cancer, etc. A prime limit factor to the administration of Adriamycin is cardiotoxicity. The most serious side effect of Adriamycin administration is myocardial degeneration causing congestive heart failure. This acute cardiomyopathy may cause acute left ventricular dysfunction, arrhythmia and myocardial infarction. Adriamycin induced cardiomyopathy is thought to be permanent and rapidly progressive. Late cardiomyopathy develops in weeks, months or even years. Some patients were reported to have developed progressive cardiomyopathy two and two-half years after receiving this drug.

On the other hand, a great development in the oncology, enabled by progression of molecular and cellular technologies throughout the culminating in 1990s was transformed from killing both cancer and normal cells to induce diverting cancer cells to resemble normal cells and do not injure the normal cells.

Many reports indicated that the side effects of anticancer drugs, which extracted from plants, are lower than chemical and antibiotic's anticancer drugs. Therefore, the development of plant drug has progressed very fast now. Taxol, for example, is a novel anticancer plant drug isolated from the needles and bark of the western yew, Taxus brevifolia. It is the prototype for a new class of antitumor drugs, which are characterized by their capacity to promote the assembly of microtubules. Clinical trials conducted in the late 1980s and early 1990s demonstrated impressive clinical activities against advanced ovarian and breast cancer. Taxol, however, still has some side effects, for example, myelosuppression, diarrhea, emesis, oligospermia, cellular depletion in lymphoid tissues, changes in serum hepatic enzymes, and elevations in cholesterol and triglycerides were observed. More important, taxol has two big problems. The first problem is that natural source of taxol is very limited. And the second problem is that taxol is a poor-water soluble. Vehicles for parental administration on taxol cause serious side effects.

HHT was used with success in the U.S. and China in the treatment of acute and chronic leukemia (Susan O'Brien et al: Blood, vol 93, No 12, 1999, pp 4149-4153). HHT is extracted from skins, stems, leaves and roots of Cephalotaxus fortunei Hook and other related species, such as Cephalotaxus sinensis Li, C. hainanensis, and C. wilsoniana. However, growth of these plants is slowly and concentration of HHT in these plants also is extremely low (typically 0.01%). Alternative sources are needed to meet the increasing demand. So far, the total chemical synthesis of HHT is not available for commerce and industry. For the reasons given above, cell culture and partial synthesis of HHT is an important new source.

HHT is a new anticancer drug because it has lower side effects, and can divert human cancer cells to closely normal cells, induce apoptosis and inhibit growth of cancer cells. More important, if HHT combined with special botanical drugs it treats cancer more effectively and is more safely.

In June 2004, FDA issued the “Guidance for Industry Botanical Drug Products” (“Guidance”), which explains when a botanical drug can be marketed under an OTC drug monograph and when FDA approval of a NDA is required for marketing. It strongly shows the FDA to open the U.S market to qualified botanical drugs.

Guidance states that applicants may submit reduced documentation of nonclinical (preclinical) safety and of chemistry, manufacturing, and controls (CMC) to support an IND for initial clinical studies of botanicals that have been legally marketed in the United States and/or a foreign country as botanical drugs or dietary supplements without any known safety concerns.

For botanicals legally marketed under the health food in the US before, FDA requires little new CMC or toxicological data needed to initiate such trials, as long as there are no known safety issues associated with the product and it is to be used at approximately the same doses as those currently or traditionally used or recommended. FDA's Guidance opens a great opportunity for developing botanical drugs in the U.S.

Also, the cost for research and development of botanical drugs is much lower than chemical drugs. Approximately 20% of pharmaceutical revenue is spent on the search for new products. One in 1000 compounds will advance from discovery to preclinical screening, one-half of those will drop prior to clinical trials, and four-fifths of those remaining will drop in clinical trials. As such, pharmaceutical discovery is an industry based on 0.1% success. Now, estimates concerning drug development costs are $800 million, roughly. Therefore, developing botanical drugs have a great economic value.

For the reasons given above, developing safe anticancer drugs, which extracted from plants or anticancer botanical drugs, are very important.

### DETAILED DESCRIPTION OF THE INVENTION

The present invention disclosed that HHT induced diverting of cancer cells, including leukemia cells and gastric and other cancer cells to resemble normal cells. HHT also inhibits growth and induced apoptosis of cancer cells. Diverting cancer to normal cells and apoptosis is important goal of cancer therapy.

The present invention provides a new method for extracting HHT by culture cells and plant tissues. New culture method for production of HHT provides cells with high HHT productivity and special elicitation which caused by derivative of gibberellin. The new culture method significantly increased the content HHT. In general, many useful products of natural ingredients by plant cell stored within the cells thus making their efficient product very difficult. The amounts and rates of production of these useful products are very low. Our method induces secretion of HHT by cells into the culture. Therefore, present invention provided a new method for production HHT on large scale. More important is elicitor for producing of HHT in culture. The present invention disclosed the gibberellin's derivative is effective elicitor.

Phenylalanine and tyrosine are precursor for biosynthesis of HHT. This scheme predicted that HHT should be generated from two molecules of phenylalanine or tyrosine. Therefore, added phenylalanine and tyrosine into culture system is very important for increasing producing of HHT.

Semi-synthesis HHT is another new source. HHT can be synthesis from large and inactive natural alkaloids.

The present invention disclosed that HHT combined with some botanical drugs could induce cancer cells to resemble normal cells.

To add some effective drugs combined with HHT can significantly increase anticancer effects of HHT. These drugs include Matrine (MAT), Guanzhongsu (GU), Maidongsu (MU), and Indirubin (IND). The experimental data showed that above drugs have strong synergisms effects for treating leukemia and other cancer cells and more safe. These new drugs are synergists for HHT.

The following specific examples will provide detailed illustrations of methods of producing relative drugs, according to the present invention and pharmaceutical dosage units containing demonstrates its effectiveness in treatment of cancer cells. These examples are not intended, however, to limit or restrict the scope of the invention in any way, and should not be construed as providing conditions, parameters, reagents, or starting materials which must be utilized exclusively in order to practice the present invention.

### EXAMPLE 1

Production of HHT by Culture Cells

So far, HHT is extracted from stems and skins of Cephalotaxus species. However, growth of plant of Cephalotaxus species is very slow and concentration of HHT in plant is extremely low. Furthermore, it is difficult to harvest the plants because of their low propagation rate and the danger of drastic reduced in plant availability. Also, cost of total chemical synthesis of HHT is very expensive and is not available for commerce now. For the reasons given above it is more difficult to obtain Cephalotaxus on a large scale for long time. Therefore, Cephalotaxus cell cultures are one of best methods for obtaining HHT.

In this present invention, special elicitation is disclosed and it will significantly increase production of HHT.

Method(s)

Cell and tissue culture

Parts of stems, leaves, skins or roots of Cephalotaxus species were surface disinfected by treatment in 70% ethanol for 10 minutes and followed by 0.1 HgCl₂ for 3 minutes. Plant materials were washed five times for 10 minutes each by sterilized water. Parts of plant were cut into small pieces (0.5-1 mm) and put pieces to Murashige and Skoog's (MS) medium and supplemented with derivative of gibberellin, phenylalamine (PHE), tyrosine (TYR) naphthaleneacetic acid (NAA), Kinetin (3 mg/L), and 3% sucrose (w/v). PH of medium was adjusted to 5.7˜5.8. Agar (10 g/L) added to medium. Callus tissues are collected from agar media and suspension cultured cells were harvested by filtration and cultured in MS medium.

The cultures were kept in a culture room at 26° C.±1 ° C. Friable callus tissues were obtained. Approximately 100 of the callu was inoculated into 4 L of MS liquid medium containing sucrose, derivative of gibberellin, PHE, TYR, NAA and Kinetin. Then callus tissues were cultivated 26° C. for 35 days on rotary shaker operated at 120 rpm in the dark. Cells were subcultured into fresh medium of same composition every 2 weeks and maintained at 120 rpm at 26°±1° C. Packed cell volume (PCV), fresh weight (FW), dry weight (DW), concentration of HHT and concentration of sugar were determined every 5th day. The cells were harvested and dried.

In general, callus and suspension cultures of cephalotaxus species grow very slow and no production of free or esterified HHT. However, according to the present invention, addition of gibberellin and other elicitors to cultures cause a drastic increasing in HHT after 10 days of incubation. For example, in control group (no gibberellin), HHT in cultured cells is 0.020 mg/g dry weight, but in treatment group (addition of gibberellin) HHT is 0.050 mg/g dry weight. It has resulted in plant cell culture systems that producing HHT at concentration higher than those produced by the mother plant. Obviously, the present invention provided a new commercial and economic method for producing HHT.

### EXAMPLE 2

HHT Extracted from Plant Tissue

Extraction of HHT has several major methods which including extraction by organic solvent, chromatograph and adjust pH.

HHT was extracted from plant tissue culture, plant cells or the skins, stems, leaves, seeds and roots of Cephalotaxus species.

1 kg of ground Cephalotaxus fortunei Hook was extracted with 8 liters of 90% ethanol at room temperature for 24 hrs. Filtered the solution to yield a filtrate A and filtercake. Percolated the filtercake with ethanol and filter again to yield filtrate B. Combined A and B, and distilled under reduced pressure to recover ethanol and an aqueous residue. To this residue, added 2% HCl to adjust the pH to 2.5. Separated the solids from the solution by filtration to yield a filtrate C. Washed the solids once with 2% HCl and filtered to yield a filtrate D. Combined C and D and adjusted the pH to 9.5 by adding saturated sodium carbonate solution. Extracted the alkaline filtrate with chloroform and separated the chloroform layer from the aqueous layer. Repeated this extraction process five times. Combined all the chloroform extracts and distilled at reduced pressure to recover chloroform and alkaloid as a solid residue respectively. The solid alkaloid was then dissolved in 6% citric acid in water. The solution was divided into three equal portions. These were adjusted to pH 7, 8 and 9 by adding saturated sodium carbonate solution. The portions having pH 8 and 9 were combined and extracted with chloroform. The chloroform extracts were distilled under reduced pressure, whereby chloroform was removed and recovered and crude HHT was obtained. The crude HHT was dissolved in pure ethanol and crystallized. The crystals were refined by recrystallization in diethyl ether.

The portion having a pH of 7 passed through a liquid chromatographic column packed with alumina of diameter to height 1:50. The column was finally flushed with chloroform and followed by chloroform-methanol of 9:1 mixture. The resulting alkaloids were mixture of HHT. The mixed HHT was then separated from each other by countercurrent distribution employing chloroform and pH 5 buffer. The first fraction of the countercurrent distribution was HHT. HHT was purified by crystallization in methyl alcohol and dried under vacuum.
HHT has the following chemical structure:
Yield: 0.02%.
Melting point: 144°-146° C.
Infrared spectrum: 3500, 3400, 1665, 1030 and 940 cm-.
Ultraviolet spectrum: λspeak alcohol mμ (logγ): 240 (3.55), 290 (3.61).

### EXAMPLE 3

Semi-Synthesis of HHT

HHT shows a significant inhibitory activity against leukemia and other cancer. Concentration of HHT, however, has only 0.01 % in natural sources. Cephalotazine (CEP) is major alkaloids present in plant extracts and the concentration of Cephalotaxus has about 1%. Therefore, concentration of CEP is about 100 times higher then HHT in plant sources. But CEP is inactive. Therefore, synthesis of HHT from CEP will increase large additional sources of HHT.

(1) Extraction of CEP

1 kg of dried stems or leaves or roots of Cephalotaxus species were milled, placed in a percolator, along 8 L of 95% of ethanol, and allowed to stand 24 hours. The ethanol was recovered under reduced pressure (below 40° C.). 2 L of 5% tartaric acid was added to concentrated ethanol solution. The ammonia water was added to the acidic solution and adjusted pH to 9. The solution of pH 9 was filtered and yielded a filtrate. The filtrate was extracted with CHCl₃. CHCl₃ was recovered under reduced pressure and residue was obtained. The residue was chromatographed packed with alumna and eluted by CHCl₃-MeOH (9:1). Eluate was concentrated under reduced pressure. Residue was dried under vacuum. The product is CEP.

(2) Synthesized HHT from CEP

Materials and Methods

Melting points were determined on a Fisher-Johns apparatus. Infrared spectra were obtained on a Perkin-Elmer 567 infrared spectrophotometer or on a Beckman 4230 IR spectrophotometer. Peak positions were given in cm⁻¹. The IR spectra of solid samples were measured as potassium bromide dispersions, and the spectra of liquids were determined in chloroform or carbon tetrachloride solutions. NMR spectra were measured on a Varian A-60, Perkin-Elmer R-32, Varian EM-390, or Brüker WH-90 NMR spectrometer. Chemical-shift values were given in parts per million downfield from Me₄Si as an internal standard. Mass spectra were run on an AEl MS-12 Finnigan 3300, or CEC21-110B mass spectrometer.

Preparative thin-layer chromatography was accomplished using 750-μm layers of aluminum oxide HF-254 (type E), aluminum oxide 60 PF-254 (type E), silica gel HF-254 (type 60 PF-254), or silica gel GF-254. Visualization was by short-wave ultraviolet light. Grace silica gel, Grade 923, and Woelm neutral aluminum oxide, activity III, were used for column chromatography. Analytical thin-layer chromatography was run on plastic sheets precoated with aluminum oxide F-254 neutral (type T), 200-μm thick, and on Polygram Sil G/UV254 (silica gel), 250 μm on plastic sheets. Visualization was usually by short-wave ultraviolet light, phosphomolybdic acid, or iodoplatinate.

a-ketoester-harringtonine

10 mg of Benzene-a-acetone Na was put into 100 ml of benzene. Mixture was stirred at room temperature then was dissolved in 100 ml of pyridine and stirred at 0° C. Oxalic chloride was added from a dropping funnel to solution of pyridine. Stirring was continued while the solution warmed to room temperature and stand overnight. Excess reagent was removed. This solution was dissolved in CH₂Cl₂ and cooled to near 0° C. in an ice water bath. 50 mg of CEP, 25 ml of CH₂Cl₂ and 25 ml of pyridine were added to cold CH₂Cl₂ solution. Manipulations were done in a dry N₂ atmosphere and all glassware heat-dried just before use. The suspension was stirred at room temperature and overnight. The mixture (2) was washed with 10% Na₂CO₃ and saturated aqueous NaCl, then dried with auhydrous magenesium sulfate, and filtered and the solvents were removed in vacuo. Evaporation provided as an amorphous solid a-ketoester-harringtonine (mp 143˜145° C.).

Semi-synthesis of HHT

100 ml of CH₃CHBrCOOEt and activated zin dust and THF were added to the α-ketoester-harringtonine (at −78° C.) for 6 hours followed by slow warming to room temperature with stirred. The reaction mixture was diluted with 100 ml CHCl₃ and 100 ml H₂O and solid Na₂CO₃ was added. CHCl₃ was evaporated under reduced pressure and residue was obtained.

The residue was purified by chromatography on alumina. The column was finally flushed with chloroform and followed by chloroform-methanol (9:1). The solvents were recovered under reduced pressure to provide as a solid. Solid was dissolved in pure ethanol and crystallized. The crystals were refined by recrystalization in diethyl ether. The crystals dried under vacuum. The product is HHT.
[α]_{D}-119° (C=0.96),
MSm/e (%): 689 (M⁺, 3), 314 (3), 299 (20), 298 (100), 282 (3), 266 (4), 20 (3), 150 (8), 131 (12), 73 (18)

### EXAMPLE 4

Extraction of Guanzhongsu

The roots and stems of Dryopteris crassirhizoma Nakai or Osmundajaponica Thunb were dried and powdered.

10 liters of water was added to 1 kg of dried powders. The solution was heated to boil and simmered one-half hours after boiling. This extraction was repeated once and the two extracts combines and filtered. The filtrate was concentrated to 5 liters and then concentrated under reduced pressure to approximately 500 ml. 95% of ethanol was added to the concentrate to a final ethanol concentration of 75%. The solution of 75% ethanol was filtered the residue (1) and filtrate saved. The filtrate was concentrated under reduced pressure to 150 ml. 100 ml of water added to 150 ml of concentrated filtrate then concentrated under reduced pressure to 150 ml and allowed to stand at 4° C. The solid precipitate (1) was separated then filtered with vacuum filter and filtrate (1) saved. Residue (1) was extracted with 1 N sodium hydroxide. Filtered and filtrate (2) were saved. Sodium hydroxide was added to filtrate (1) to a final sodium hydroxide concentration of 1 N and combined with filtrate (2) then neutralized with 1N of HCl. Neutralized solution allowed to standing at 4° C. Solid precipitate (2) was separated and combined with solid precipitate (1). Solid precipitate was dried under vacuum and powered.

### EXAMPLE 5

Extraction of Maidongsu

1000 g of Maidongsu (Ophiopogonjaponica Ker-Gawl) powder were soaked in 2000 ml of 75% ethanol at room temperature for 24 hours. The mixture was filtered and the filtercake powder was refluxed twice for 2 hours with 2000 ml of 75% ethanol and filtered.

The filtrates were combined and distilled on a steam bath at 17 mm Hg absolute, whereby wet ethanol was evaporated and an aqueous still residue was obtained. This still residue was extracted with 500 ml of diethyl ether four times to remove lipids. Other solvents such as petroleum ether might be used.

To this aqueous raffinate were added 500 ml of n-butanol and the mixture was evaporated to dryness at 17 mm Hg absolute, whereby about 30 g of powder residue were obtained. This is Maidongsu.

### EXAMPLE 6

Extraction of Matrine (MAT)

MAT was Extracted from Root of Sophora flavescens Ait.

1 kg of ground plant was extracted 5 liters of methanol for 12 h at room temperature. The resulting methanol extract was filtered. Methanol was then recovered under reduced pressure distillation. A distillated residue was dissolved in 300 ml of HCl and adjusted the PH to 3.5. NaOH was added to HCl solution and adjust pH to 13. Solution of pH 13 extracted by CH₂Cl₂ and then CH₂Cl₂ was recovered under reduced pressure distillation. The residue was dissolved in CHCl₃. Diethyl ether was added to CHCl₃. The mixture was filtered. The filtrate was concentrated to syrup under reduced pressure distillation. The residue passed through a chromatographic column packed with alumina again. The column was eluted with oil ether-acetone. The elution was concentrated under reduce pressure. The residue was passed through a chromatographic column packed with alumina again. The column was eluted with benzol-oil ether. The organic solvent was recovered under reduced pressure and residue obtained. The acetone was added to residue and crystallized. The crystals were refined by recrystallization in acetone. The crystals were dried under vacuum and were found to have a meting point of 76° C. and [α]_{D}+39.1° (H₂O).

### EXAMPLE 7

Extraction of Indirubin (IND)

Indirubin was extracted from stems and leaves of Baphicanthus cusia (Ness) Bremek, or Isatis tinctoria L, or Isatis indigotica Fort, and Polygonum tinctorium Ait.

Isolation of Isatin B

10 kg of dried powder of plant was extracted with hot water 20 liters. The extract was filtered. The filtercake extracted with methanol (10 liter). Methanol was recovered under reduced pressure and residue obtained. The residue was extracted with chloroform (5 liter). The chloroform was recovered and the chloroform residue was chromatographed on silica gel G (1 kg), using chloroform as developing solvent. The eluate was concentrated and rechromatographed on silica gel G (500 g) with chloroform as solvent. The IND was crystallized from chloroform and recrystallized and then dried under vacuum.
Melting point: 356˜358° C.
UVλ^{CHCl3} nm: 242, 292, 362, 540
IRλ^{KBr} cm⁻¹: 3345, 1670, 1620, 750.

### EXAMPLE 8

Effect of HHT and in Other Drugs on Differentiation of Human Leukemic Cells

Methods

Cell Lines

HL-60 cells were established from a patient with acute myeloid leukemia. The cells were cultured in culture flasks with RPMI plus 10% FCS.

Studies of Induction of Differentiation

Differentiation of HL-60 cells was assessed by their abilities to produced superoxide as measured by reduction of NBT, by NSE staining and by morphology as detected on cytospin preparations stained with Diff-Quick stain Set, and by analysis of membrane-bound differentiation markers with two-color immunofluorescence. Briefly, cells were preincubated at 4° C. for 60 min in 10% human AB serum and then with FITC-conjugated mouse IgGI isotype control. Analysis of fluorescence was performed on a flow cytometer.

Clonogenic Assay in Soft Aga.

HL-60 cells were culture in a two-layer soft agar system for 10 days without adding any growth factors as described previously, and colonies were counted using an inverted microscope. The analogues were added to the agar upper layer on day 0. For analysis of the reversibility of inhibition of proliferation, the cells were cultured in suspension culture with and without HHT or other drugs. After 60 hours, the culture flasks were gently jarred to loosen adherent cells, the cells were washed twice in cultured medium containing 10% FCS to remove the test drugs, and then the clonogenic assay was performed. NBT% indicated percentage of normal cells.

These results were periodically confirmed by fluorescence microscopy and by DNA fragmentation.

TABLE 1
Effect of drugs on cellular diversion of human leukemic cells

| Group | NBT (%) |
|---|---|
| N | 98 ± 12 |
| C | 5.0 ± 0.6 |
| HHT | 65 ± 7.0* |
| HHT + GU | 78 ± 9.2* |
| HHT + MU | 75 ± 7.2* |
| HHT + IND | 82 ± 9.8* |
| HHT + MAT | 79 ± 9.5* |

N: Normal cells;
C: Human leukemic cells
*P < 0.01 compared with control group. Concentration of Mu, GU, MAT, and IND is 50 ng/ml.
NBT (%) is index of normal cells. The higher NBT (%) means higher normal cells.

Data of Table 1 showed that HHT could significantly induce diversion of leukemic cells to normal cells. And other drugs increasing induce diversion of leukemic cells to normal cell be HHT.

Results of other methods are similar data of Table 1.

### EXAMPLE 9

Effect of HHT on Cellular Diversion of Gastric Cancer Cells

The gastric cancer cells and normal cells were cultured in PRMI 1640 medium supplement with 10% FCS serum. Other method is similar to example 8.

TABLE 2
Effect of HHT on diversion of gastric cancer cells

| Group | NBT (%) |
|---|---|
| N | 95 ± 14 |
| C | 8 ± 1.2 |
| HHT | 58 ± 8.9* |
| HHT + GU | 72 ± 8.9* |
| HHT + MU | 70 ± 8.1* |
| HHT + IND | 72 ± 8.0* |
| HHT + MAT | 65 ± 7.5* |

*P < 0.01 compared with control group.

Data of Table 2 showed that HHT could significantly induce diversion of gastric cancer cells to normal cells. And other drugs increasing induce differentiation of gastric cancer cells by HHT.

### EXAMPLE 10

Effect of HHT and Other Drugs on Apoptosis of Cancer Cells

Methods

Human leukemia cells (HL-60) were grown in RPMI Medium 1640 supplemented with 10% (v/v) heat-inactivated FBS (56° C. for 30 min) at 37° C. in a humidified 95% air/5% CO₂ atmosphere. Cells were seeded at a level of 2×10⁵ cells/ml. Cells were allowed to attain a maximum density of 1.2×10⁶ cells/ml before being passed by dilution into fresh medium to a concentration of 2×10⁵ cells/ml.

Cell pellets containing 5×10⁶ cells were fixed with 2.5% glutaraldehyde in cacodylate buffer (pH 7.4), dehydrated through graded alcohol, and infiltrated with LX-112 epoxy resin. After overnight polymerization at 60° C. 1-μm sections were cut with glass knives using a microtome. The sections were stained with 1% toluidine blue and coverslipped. In addition, experimental examples were stained with May-Grunwald-giemsa stain for the demonstration of apoptosis.

Determination of Apoptosis

Method (1): Apoptosis was Quantitated by Flow Cytometry

Cells (2.5×1060 were incubated in 10 ml IMDM plus 105 heat-inactivated fetal calf serum. Samples were incubated for 24 hours with various concentrations of drugs. Control samples received the same amount of media, without drug addition. After 24 hours of incubation the samples were pelleted and fixed in ethanol 705 for 15 minutes at 4° C.; after three washes in PBS, the cells were treated with RNase I 0.5 mg/ml for 15 minutes at 37° C. The cells were harvested by centrifugation and resuspended in 50 μg/ml propidium iodide in PBS. Analysis (upon acquisition of 10,000-20,000 events) was performed on a FACscan flow cytometer with the FL2 detector in logarithmic mode, using Lysis II software (Becton Dickinson). Apoptotic cells were located in the hypodiploid region of the histogram, due to chromosome condensation and fragmentation.

For evaluation of apoptosis by flow cytometry, cells were fixed and permeabilized in 1% paraformaldehyde and ice-cold 70% ethanol. Digoxigenin-dUTP was incorporated at the 3′OH ends of the fragmented DNA in the presence of terminal deoxynucleotidyltranserase, and the cells were incubated with FITC-labeled anti-digoxigenin-dUTP and with propidium iodide. Green (apoptotic cells) and orange (total DNA) fluorescence were measured with a FACScan flow cytometer and analyzed with LYSIS II and CELLFIT programs. Data were analyzed by Student's t-test. P values were considered significant when <0.05.

Method (2): DNA electrophoresis

Untreated and treated HL-60 cells collected by centrifugation, washed in phosphate buffered saline and re-suspended at a concentration of 5×10⁶ cells and 0. 1% RNase A. The mixture was incubated at 37° C. for 30 min and then incubated for an additional 30 min at 37° C. Buffer was added and 25 μl of the tube content transferred to the Horizontal 1.5% agarose gel electrophoresis was performed at 2 V/cm. DNA in gels visualized under UV light after staining with ethidium Bromide (5 μg/ml).

DNA fragmentation assays: DNA cleavage was performed, quantitation of fractional solubilized DNA by diphenylamine assay and the percentage of cells harboring fragmented DNA determined by in labeling techniques. For the diphenylamine assay, 5×10⁶ cells were lysed in 0.5 mL lysis buffer (5 mmol/L Tris-HCl, 20 mmol/L DTA, and 0.5% Triton X-100, pH 8.0) at 4° C. Lysates were centrifuged (15,000g) for separation of high molecular weight DNA (pellet) and DNA cleavage products (supernatant). DNA was precipitated with 0.5 N perchloric acid and quantitated using diphenylamine reagent. The cell cycle distribution was determined 4 hours after addition of drug and represents mean ±SD of 5 independent experiments.

Method (3)

Apoptosis of HL-60 cells was assessed by changes in cell morphology and by measurement of DNA nicks using the Apop Tag Kkt (Oncor, Gaithersburg, Md.). Morphologically, HL-60 cells undergoing apoptosis possess many prominent features, such as intensely staining, highly condensed, and/or fragmented nuclear chromatin, a general decrease in overall cell size, and cellular fragmentation into apoptotic bodies. These features make apoptotic cells relatively easy to distinguish from necrotic cells. These changes are detected on cytospin preparations stained with Diff-Quick Stain Set. Apoptotic cells were enumerated in a total of about 300 cells by light microscopy.

TABLE 3
Effect of drugs on apoptosis of cancer cells (1)

Apoptosis (%)

| | Normal cells | Human leukemia cells |
|---|---|---|
| No drug | 8.1 ± 2.0 | 4.0 ± 0.9 |
| HHT (10 ng/ml) | 16.5 ± 2.0* | 33.8 ± 4.8** |
| HHT (50 ng/ml) | 18.5 ± 2.5* | 42.8 ± 5.2** |
| HHT (100 ng/ml) | 22.0 ± 3.5* | 60.2 ± 8.9** |
| HHT (500 ng/ml) | 29.5 ± 5.2* | 92.0 ± 10.8** |

*P < 0.01 compared with group of normal cells.
**P < 0.01 compared with group of human leukemia cells

TABLE 4
Effect of drugs on apoptosis of cancer cells (2)

Apoptosis (%)

| | Normal cells | Human leukemia cells |
|---|---|---|
| HHT (50 ng/ml) | 18.5 ± 2.5 | 42.8 ± 5.2 |
| HHT + GU | 12 ± 2.5* | 58 ± 7.9* |
| HHT + MU | 10 ± 2.0* | 51 ± 6.8** |
| HHT + MAT | 15 ± 2.4 | 60 ± 8.2* |
| HHT + IND | 9 ± 1.8* | 52 ± 5.9** |

*P < 0.01 compared with group of human leukemic cells
**P < 0.05 compared with group of human leukemic cells

Data showed that HHT could significantly induce apoptosis of cancer cells. The effects of various drugs combination with HHT (50 ng/ml) on normal and human leukemia cells are showed in the Table 3-4. Other drugs exerted a synergistic effect with HHT in increasing apoptosis of human leukemia cells and decreasing apoptosis of normal cells (Table 4). It means that GU, MAT, MU and IND exerted an additive effect with HHT in treating human leukemia and protective normal cells.

### EXAMPLE 11

Effects of Drug on Tumor Cells Proliferation

Materials and Methods

Human tumor cell lines: Hela leukemia HL-60, malignant melanocarcinoma B 16, oral epidermoid carcinoma (KB), lung carcinoma (A549), breast carcinoma MCF-7, adenocarcinoma of stomach.

Animal tumor cell lines: Walker carcinoma, LLC-WRC-256, malignant melanoma (RMMI 1846), 3T3, and S-1 80 sarcoma (CCRF-180). All lines were routinely cultured in the RPMI1640 medium supplemented 20% fetal calf serum. The experiment was carried out in 96 microplate, each well had 5×10⁵ cells and given desired concentration of 1 μg/ml (1×10⁻⁶ g/ml) drug. Then the plate was incubated at 37° C. in an atmosphere of humidified air enriched with 5 percent carbon dioxide for 72 hours. Inhibition percent rate of tumor cell proliferation was obtained according to the bellow formula.

$$Inhibition percent rate = \frac{Control - Test}{Control} \times 100 \%$$

Results

TABLE 5
Effect of drugs on inhibiting growth cancer cells

| Group | Inhibition (%) |
|---|---|
| No drug | — |
| HHT | 73.8 ± 8.1 |
| HHT + GU | 74 ± 12.9 |
| HHT + MU | 76 ± 19.8 |
| HHT + IND | 89.9 ± 12.5* |
| HHT + MAT | 58 ± 16.8* |

*P < 0.01 compared with HHT group.

Data of Table 5 showed that HHT could significantly inhibit human cancer cells proliferation and other drugs could increase effect of inhibiting cancer cells by HHT.

### EXAMPLE 12

The Effect of HHT and Other Drugs on the Growth of Transplanted Tumor

Experimental Procedure

Male mice, weight 20-22 g, were used in the experiment. 1×10⁷ tumor cells were injected to mouse and other drugs injected intraperitoneally began second day. All mice were sacrificed on the 12th day, isolated the tumor and weighed and calculated the inhibition rate of tumor weight.

Results

The effect of other drugs and HHT on the growth of animal transplanted tumor as illustrated by the Table 6.20 mg/kg of other drugs could inhibit the growth of L615 transplanted tumor.

TABLE 6
Effect of drugs on inhibition of transplanted tumor

| Group | Inhibition (%) |
|---|---|
| No drug | — |
| HHT | 68 ± 7.2 |
| HHT + GU | 75.2 ± 9.8 |
| HHT + MU | 74.8 ± 10.5 |
| HHT + IND | 89.8 ± 12.0* |
| HHT + MAT | 82.7 ± 10.5* |

*P < 0.01 compared with HHT group.

Data of Table 6 showed that HHT could significantly inhibit animal transplanted tumor and other drugs could increase effect of inhibiting cancer cells by HHT.

### EXAMPLE 13

The Effect of Drug on Decreasing of Tyrosine Kinase

The development of cancer cells can be viewed as a defect in the normal process of differentiation and disorder balance between proliferation and maturation that occurs in normal cells. The expression of oncogenes plans a very important role in regulate cellular proliferation. The tyrosine kinase (TK) is a protein product of expression of oncogenes. The TK catalyze the transfer of phasphate from ATP to the hydroxyl residues on protein substrates. Activity of the TK is essential for the malignant transformation of cells.

In subsequent years, a number of oncogenes have been found to code for TK. Such as src, yes, fgr, abl, erbB, mos, neu,fms, fps, ros and sis are considered to act through tyrosine kinase activity. TK activity is strongly correlated with the ability of retroviruses to transform cells. Also, maturation with reduced TK activity has lower transforming efficiency. Transformation of the HL-60 leukemia cells causes the high TK activity. In fact, TK activity is enhanced in many human cancers, such as breast carcinomas, prostate cancer cells, colon cancers, and skin tumor. The results of a lot of experiments indicated that tyrosine phosphorylation is an important intracellular mediator of proliferation and differentiation. Mature of cells possess relatively low levels of TK activity. Similar TK activity is also related with the cellular receptors for several growth factors such as EGF, platelet-derived growth factor, insulin, and growth factor.

In general, very low levels of TK are expressed in normal cells and high levels of TK are expressed in cancer cells. Many evidences have been accumulated that the dysfinction of cellular oncogenes is a cause of human cancers. Therefore, a drug, which inhibits the activity of TK, can provide a new way to overcome cancer. In other words, the development of effective inhibitors of TK can be used for the treatment of cancer. Materials and Methods

[³²P]ATP and other isotopes were purchased form Amersham Corp. All other chemicals were reagent grade obtained from commercial suppliers.

Cells: L1210 and P388 cells were grown at 37° C. on medium RPMI-1640 without antibiotics and supplemented with 10% horse serum. Cultures were diluted daily to 1×10⁵ cells/ml with fresh growth medium. From a culture initiated with cells from ascitic fluid obtained from a mouse 5 days after implantation with in vivo-passage leukemia, a stock of ampoule containing 10⁷ cells/ml in growth medium plus 10% dimethyl sulfoxide was frozen and stored in liquid nitrogen. Cultures were started from the frozen stock and were passage for no more than 1 month.

L1210 and P388 cells were grown at 37° C. on medium RPMI-1640 supplemented with 10% calf serum, 10,000 unit/ml of Penicillin and 10,000 unit/ml of Streptomycin. 1×10⁶/ml cells were placed in culture with different concentrations of HHT. Then the cell suspension was incubated at 37° C. in a humidified atmosphere of 5% CO₂-95% air for the indicated time. Reactions were terminated by addition of 3 ml of cold Earle's buffer. Cells were lysed, precipitated with 10% trichloroacetic acid (TCA) and filtered onto glass fiber filters. The filters were washed with phosphate-buffered saline and placed in scintillation vials, and radioactive emissions were counted.

Tyrosine kinase (TK) Assay: TK was measured by a modification of the method of Braun et al. Briefly, H-60 leukemia cells were plated at a density of 5×10⁵ cells in 60-nm dished, and divided control and treatments groups for incubation 24 hours at 37° C. with 5% CO₂. The cells were collected by scraping, washed twice with phosphate-buffered saline, and resuspended at density of 10⁶ cells/ml in 5 mM HEPEs buffer (pH 7.4). The cells were then resuspended in 1 ml of buffer containing 5 mM HEPES (pH 7.6), 1 mM MgCl₂ and 1 mM EDTA, then placed on ice bath. The cell membrane was disrupted by ultra sound and centrifuged at 1,000×g for 10 minutes. The supernatant was ultra centrifuged at 30,000×g for 30 minutes at 4° C. The pellet was resuspended in 0.3 ml of buffer containing 25 mM HEOES, centrifuged at 12,000×g for 5 minutes. The resulting supernatant was used for TK assay. Content of protein was determined. 10 μg of protein placed in 20 mM HEPES (pH 7.6), 15 mM MgCl₂, 10 mM ZnCl₂ and 5% (v/v) nonidet P-40, with or without substrate [glutamic acid (GT), mg/ml]. After 5 minutes incubation at 25° C., the reaction was initiated by the addition of 25 μM [γ³²P] ATP (3 ci/mmol). After 10 minutes, the reaction was stopped by the addition of 20 mM cold ATP. 50 μl of the mixtures were spotted on glass microfiber filter discs and washed three times with cold trichloroacetic acid (TCA), contained 10 mM sodium pyrophosphate. Air dried. Radioactivity was determined by liquid scintillation spectrometry. The net TK activity was determined after correcting for endogenous TK activity.

Results and Discussion

The present study clearly demonstrated that HHT reduction in TK activity. A concentration-dependent inhibition was seen. HHT caused a relatively strong inhibition, with inhibition 99.9% occurring at a concentration of 10⁻⁶ M.

TABLE 7
Effect of HHT on TK activity of HL-60 leukemia cells

| Drugs | Concentration (M) | % of control activity |
|---|---|---|
| No drug | — | 100 |
| HHT (1) | 10⁻⁶ | 0.6 |
| HHT (2) | 10⁻⁷ | 21.5 ± 4.0 |
| HHT (3) | 10⁻⁸ | 87.8 ± 12 |

TABLE 8
Effects of drugs on TK activity

| Group | % of control activity |
|---|---|
| No drug | 100 |
| HHT | 21.5 ± 4.0 |
| HHT + GU | 24.5 ± 4.5 |
| HHT + MU | 27.5 ± 4.3 |
| HHT + IND | 40.0 ± 6.5** |
| HHT + MAT | 31.0 ± 5.2** |

*P < 0.01 compared with HHT
**P < 0.05 compared with HHT

Data represent the mean of three experiments each done in duplicate; the range was less than 5% of the mean.

HHT inhibited the HL-60 TK activity by 0.6% at 10⁻⁶ M, 78.5% at 10⁻⁷ M and 12.2% at 10⁻⁸ M.

Table 7-8 indicated, HHT can significantly inhibit TK activity and other drugs increase the effect of decreasing TK activity of HHT.

### EXAMPLE 14

Drug Inhibited Tumor Incidence in Vivo

The capacity of tobacco-specific nitrosamine 4-(methylnitrosamino)-1-(3-pyridyl)-1-butanone (NNK) to induce tumor incidence was recognized several years ago.

Methods:

Every group had 20 mice. For treatment group, each mouse was gave Drug by injection at dose of 20 mg/kg daily. For control group, each mouse was gave same volume of physiological saline. Three days later, mice were gave 10 μmol NNK (in 0.1 ml saline) by i.p. injection. Sixteen weeks after these treatments the mice were killed and pulmonary adenomas were counted. The statistical significance of bioassay data was determined by student's test.

TABLE 9
Effect of Drug on NNK-induced lung tumorigenesis

| Group | Tumor incidence (%) |
|---|---|
| No drug | 100 |
| HHT | 35.8 ± 4.5 |
| HHT + GU | 21.7 ± 4.8* |
| HHT + MU | 26.5 ± 4.9* |
| HHT + IND | 20.6 ± 3.8* |
| HHT + MAT | 28.5 ± 3.9* |

*P < 0.05 compared with HHT group

Data of Table 9 indicated that HHT has a significant inhibitory effect against lung tumor and could decrease tumor incidence. Therefore, HHT could prevent cancer. Other drugs could increase the effect of HHT.

### EXAMPLE 15

Effect of Drug on Peroxidation

The peroxidized lipids were deleterious to health as they induce cross-linking of DNA and proteins damage. The oxidized low-density lipoproteins were efficiently phagocytosed by macrophages through scavenger receptors.

Methods:

The liver microsome (lmg protein) in 0.15 M KCl, 0.25 M Tris-HCl buffer, pH 7.5, 2 mμ ADP, 10 μM FeSO₄ and Drug B (50 μM) were incubated at 37° C. for 5 min. The reactions were terminated after 30 min by added 2 ml thiobarbituric acid (TBA) and content of the malondialdehyde (MDA) formed was determined.

TABLE 10
Effect of Drug on lipid peroxidation

| Group | Nmoles MDA/mg protein |
|---|---|
| No drug | 25.4 ± 0.3 |
| HHT | 8.5 ± 1.0 |
| HHT + IND | 4.0 ± 0.6* |
| HHT + MAT | 6.5 ± 0.9* |
| HHT + MU | 7.1 ± 1.8* |
| HHT + GU | 6.8 ± 1.7* |

*P < 0.01 compared with HHT group
Results are mean ± SD value from 20 rats.

Data of table 10 indicated that HHT could significantly inhibit lipid peroxidation. Decreasing lipid peroxidation could prevent cancer. Other drugs could increase the effect of HHT.

### EXAMPLE 16

Effect of Drug on Inhibition of Carcinogen-DNA Binding

Inhibition of carcinogen-DNA binding was one of biochemical markers for screening potential chemopreventive agents by National Cancer Institute of the US.

Methods:

Cells were maintained in 75-cm² tissue culture flask with media supplemented with retinoic acid (0.1 μg/ml) and epinephrine (0.5 μg/ml). The cells were passage when they reached approximately 80% confluence and were maintained at 37° C. in 5% CO₂. Cells were plated at a density of 5×10⁵ cells/well in 6-well tissue culture plates and allowed to incubated for 18 h at 37° C. in 5% CO₂. Following incubation, the DNA was harvested and resuspended in Tris-EDTA buffer. RNA was removed by treating with RNase T1 and RNase A at 37° C. for 1 h. An aliquot was used for determining the DNA content by the absorbency at 260 nm, and the rest of the sample was used to determine the radioactivity; the percentage inhibition of carcinogen-DNA binding was determined by measuring the cpm/mg of DNA in the carcinogen.

TABLE 11
Effect of Drug on inhibition of carcinogen-DNA binding

| Group | Inhibition (%) |
|---|---|
| No drug | — |
| HHT | 56.8 ± 6.2 |
| HHT + IND | 40.8 ± 8.3* |
| HHT + MAT | 42.8 ± 9.6* |
| HHT + MU | 52.8 ± 8.2* |
| HHT + GU | 51.2 ± 7.5* |

*P < 0.01 compared with HHT group

Data of table 11 indicated that HHT significantly inhibits carcinogen-DNA binding and other drugs could increase the effect of HHT.

### EXAMPLE 17

Safety of HHT (1): Toxic Dose for Mice

Methods for determination of LD₅₀

Mice were used in the experiment. The animals were assigned by weight into the treatment and control groups. The animals were singly housed in hard-bottomed polypropylene cages with wood shavings. The animals had free access to food and water. Lighting was controlled on a 12 hours light; 12 hours dark cycle, (lights on 8 a.m.; lights off 8 p.m.). The housing facility temperature was maintained at 20°±2°. Humidity was maintained between 50-70%.

Parameters Assessed

Bodyweight, food and water intake. Prior to commencement of the study, all animals were weighed and assigned to groups, ensuring all groups had a similar mean weight. The body-weight of each animal was recorded prior to drug administration, as was food and water. These values were recorded again 24 hours later and the body- weight change, food and water intake was calculated as the difference between these three measurements.

Home cage activity. Animals were singly housed in a home cage monitor and their activity monitored during the nocturnal period (8 p.m.-8 a.m.), throughout the study. The cage in which the animal is housed (home cage) is placed into a compartment on a rack. On the top of each compartment there is a passive infra-red (PIR) sensor. The sensor is powered by a 10 volt direct current power supply. This splits the infra-red beam into 16 zones which radiate across the floor of the cage. The 24 sensors are connected by separate switch inputs to an interpak 2 interface. The whole system is controlled by the home cage activity monitor software package.

The data are listed as below.

LD₅₀: The LD₅₀ of HHT in mice (I.P.) was found to be 3.17±0.19 mg/kg.

Toxic doses for mice: In 38 normal mice after injection of HHT of 2 mg/kg/day×5 with the observation period of 5 days about 50% of the mice died.

### EXAMPLE 18

Safety of HHT (2): Toxic Dose for Dog

Forty dogs were used. Body weight, food consumption, general observations, laboratory tests, and postmortem examinations were determined.

Twenty normal adult dogs which weight approximately 10 kg±1 kg used in experiments. Toxic doses for dogs: 0.15 mg/kg/days×7 and 0.30 mg/kg/days×7 of HHT was injected as the toxic doses.

For HHT treatment group, at least eight dogs that died early in the observation period and eight dogs that died late were examined. After injection of lethal doses the major target organs involved in toxicity in dogs produced by 7 day treatments with HHT were limited to G.I. tract, heart and hematopoietic organs. Most deaths were caused by cardiac dysfunction. After injection of lethal doses, hepatic toxicities of mild to moderate degree occurred only in individual cases. When such treatments were repeated for two additional courses, no additional significant toxicity was observed. However, the cardiac and hematopoietic toxicity appeared to be moderately degree.

The data of short-term toxicity in dogs for HHT were summarized as the following table.

TABLE 12
Safety of HHT

| | HHT (dose) | | | | | |
|---|---|---|---|---|---|---|
| | 0.1 mg/kg/d × 7 | | 0.2 mg/kg/d × 7 | | 0.3 mg/kg/d × 7 | |
| Reaction | 20 ♀ | 20 ♂ | 20 ♀ | 20 ♂ | 20 ♀ | 20 ♂ |
| Body weight (decreasing %) | 8.9 | 8.8 | 11.0 | 10.9 | 12.5 | 13.0 |
| Cardiovascular system | | | | | | |
| Heart rate (beat/min) | 208 | 205 | 232 | 230 | 260 | 259 |
| Abnormality of ECG, ST-T | − | − | ± | ± | + | + |
| Cardiac necrosis | − | − | − | − | ± | ± |
| Hematopoietic/hemostatic system | | | | | | |
| Hemoglobin % | 8.8 | 9.0 | 9.3 | 9.2 | 9.5 | 9.6 |
| Erythrocyte count (×10⁶/mm³) | 3.90 | 3.94 | 3.70 | 3.65 | 3.40 | 3.40 |
| Leukocyte count (× 10³/mm³) | 3.91 | 3.90 | 3.65 | 3.58 | 3.25 | 3.20 |
| Platelet count (× 10⁴/mm³) | 4.9 | 5.0 | 3.75 | 3.70 | 2.90 | 3.00 |
| Liver | | | | | | |
| Glutamic pyruvate transaminase (GPT) | 65 | 68 | 72 | 73 | 75 | 73 |
| Hepatic necrosis | − | − | ± | ± | + | + |
| Hepatic fibrosis and cirrhosis | − | − | − | − | ± | ± |
| Hepatic ecchymosis | − | − | − | − | ± | ± |
| Albumin-globulin ratio (A/G %) | 89 | 90 | 92 | 93 | 95 | 92 |
| Gastrointestinal system | | | | | | |
| Vomiting | + | + | + | + | ++ | ++ |
| Anorexia | + | + | ++ | ++ | +++ | +++ |
| Diarrhea | ± | ± | + | + | + | + |
| Marrow | | | | | | |
| Marrow depression | + | + | + | + | ++ | ++ |
| Renal/urinary system | | | | | | |
| Renal necrosis | − | − | − | − | ± | ± |
| Renal toxic nephropathy | − | − | − | − | ± | ± |
| Spleen | | | | | | |
| Spleen necrosis | − | − | − | − | ± | ± |
| Reproductive system | | | | | | |
| Seminal depression | − | − | − | − | − | − |

“±”: either positive or negative;
“+”: positive, light;
“++”: middle,
“+++”: heavy
Normal data of index are listed below.
Hemoglobin: 11.0˜13.5 g; erythrocyte count: 4.11˜5.03 × 10⁶/mm³; leukocyte count: 5.6˜10.9 10³/mm³;
Platelet count: 11.2˜34.8 × 10⁴/mm³; NPN: <46.0 mg %; GPT: <49%; A/G: 47˜65/35˜52; heart rate: 240 beat/min.

All toxicities observed were dose-dependent, completely reversible upon discontinuation of treatment. No significant delayed toxicity was noted during an observation period of 6 weeks or more. No sex related differences in qualitative toxicity of HHT were observed. In conclusion, toxicity of HHT is low.

LD₅₀ of HHT is much higher than majority anticancer drugs. The toxicology data of HHT mean that HHT is safe drug for treatment of cancer cells.

Dose: HHT was administered at a dose of 1˜5 mg/m² as infusion through venous or injection. MAT, GU, MU and IND were administered at a dose of 25˜250 mg orally.

The LD₅₀ of HHT indicated that HHT is a safe anticancer drug (it is compared with other general anticancer drugs).

### EXAMPLE 19

Safety of HHT (3): Analysis of Chromosomes

For metaphase chromosomes, kidney cell cultures were treated with colchicines (0.4 μg/ml) for 3-4 hours. The cells were then trypsinized and treated with hypotonic solution (0.075 M KCl) at 37° C. for 30 minutes. The cell suspensions were centrifuged and the pellets fixed in cold acetic acid:methanol (1:3) solution. Slides were prepared by standard air-drying method and stained with 2% Giemsa solution. The results scored by analyzing at least 200 well spread metaphases with 44±2 chromosomes for gaps, chromatid and chromosome breaks and exchanges, and association. Chromatid and chromosome aberrations were scored separately, and the total percentage was subjected to statistical analysis. Gaps were recorded but not included in the total frequency. Endoreduplication (endomitosis) was estimated from at least 500 cells/animal and expressed as a percentage.

TABLE 13
Chromosomal aberrations induced by HHT in kidney

| | | |
|---|---|---|
| Duration of | Aberration/100 cells | |
| treatment | Chromosome | % Aberrant cells |

| (months) | Breaks | Exchanges | (mean ± SEM) |
|---|---|---|---|
| Untreated | | | |
| 0.5 | nd | nd | 0.2 ± 0.1 |
| 1.0 | nd | nd | 0.3 ± 0.2 |
| 2.0 | nd | nd | 0.3 ± 0.1 |
| 3.0 | 0.1 | nd | .0.5 ± 0.3 |
| 4.0 | nd | 0.1 | 0.5 ± 0.2 |
| 5.0 | 0.1 | nd | 0.4 ± 0.2 |
| HHT | | | |
| 0.5 | 0.1 | nd | 0.5 ± 0.1 |
| 1.0 | 0.5 | nd | 0.8 ± 0.2 |
| 2.0 | 0.7 | 0.1 | 1.2 ± 0.4 |
| 3.0 | 0.9 | nd | 1.8 ± 0.7 |
| 4.0 | 1.2 | 0.1 | 2.6 ± 0.8 |
| 5.0 | 1.3 | 0.2 | 5.5 ± 0.5 |

The data of Table 13 indicated that HHT has no exchange in chromosome, no chromatid or chromosome aberrations and no significant differences in the frequency of either chromosome lesions or chromatid or chromosome aberrations with increasing age.

### EXAMPLE 20

Safety of HHT (4): Mutagenic Effect of HHT

Determination of the mutagenic and carcinogenic activity is important for estimating side effects of drug. The mutagenic activity of many drugs can only be detected with growing cells. In present study, mutagenic and carcinogenic activity of HHT is determined by Bacteria system.

The method for detecting mutagenicity of HHT, with the Salmonella system that detects the reversion of the bacteria from His⁻ to His⁺, is widely used.

Methods for detecting carcinogens and mutagens with the salmonellia mutagenicity test are highly efficient in detecting carcinogens and mutagens. Major carcinogens tested have been detected as mutagens. Salmonella mutagenicity assay is very sensitive and simply test for detecting mutagens and carcinogens. Therefore, it has been useful in a detailed study that has been made of mutagenic activity of HHT.

TAa7, TAa8, TA 100 and TA 102 are extremely effective in detecting classes of carcinogens and mutagenesis.

Methods

The bacterial tester strains used for mutagenesis testing are TA97, TA98, TA100 and TA102. Mutagenesis testing method was done as described previously ^{[77-88]}. In brief, TA97, TA98, TA100 and TA102 were grown in agar gel culture. The petri plats (100×15 mm style) contain 30 ml with 2% glucose. The agar mixture was agitated vigorously and immediately poured into plates of minimal agar. The cultures were incubated at 37° C. in a dark and 5% CO₂ in air for 48 hours. After 48 hours the colonies in both test and controls are counted. The presence of a background lawn of bacteria on the histidine-poor soft agar plate was used as an indication that gross toxic effects were absent. Mutagenicity assays were carried out at least in triplicate.

Results and Discussion

The data of experiment summarized as the following table.

TABLE 14
Mutagenesis Assay on plates

| | Dose/ | Number of His⁺ revertants/plate | | | | | |
|---|---|---|---|---|---|---|---|
| | plate | TA97 | | TA98 | | TA100 | |
| Treatment | (μg) | −S | +S | −S | +S | −S | +S |
| Spontaneous | — | 149 ± 15 | 150 ± 17 | 35 ± 4 | 36 ± 4 | 120 ± 17 | 120 ± 15 |
| 4NQO | 0.5 | 861 ± 79 | — | 338 ± 35 | — | 2301 ± 190 | — |
| HHT | 100 | 150 ± 16 | 160 ± 17 | 32 ± 4 | 34 ± 4 | 158 ± 15 | 160± |
| HHT | 10 | 160 ± 16 | 165 ± 16 | 38 ± 4 | 36 ± 3 | 162 ± 17 | 165± |
| HHT | 1 | 130 ± 11 | 150 ± 14 | 30 ± 3 | 32 ± 4 | 140 ± 13 | 152± |
| HHT | 0.1 | 120 ± 10 | 145 ± 14 | 29 ± 3 | 34 ± 4 | 148 ± 12 | 158± |

*4QO: 4-nitroquinoline-1-Oxide

The salmonella typhimurium strains TA97, TA98 and TA100 were checked using 4-nitroquinoline-1-oxide. The range of spontaneous mutation rates for the individual strains, which were considered to be acceptable, was TA97 (100-170), TA98 (20-40) and TA100 (80-150).

The data of Table 14 indicated that the number of His+ revertants/plate of HHT almost is as same as spontaneous of testing strains. On the contrary, 4NQO is mutagenic and carcinogenic agent. The number of His+ revertants/plate of 4NQO is higher than 10 times of spontaneous.

In conclusion, HHT has no carcinogenic and mutagenic action.

### EXAMPLE 21

Metabolism of [³H]-HHT

The data of metabolism of HHT provide a vital tool for understanding of HHT action including how many HHT gets to the sites where it activates pharmacological and chemotherapeutic activity and how long it remains there. The data above also help us to understand the amounts of HHT in organs and metabolites in the major organs. Metabolism of HHT is established to quantitatively dynamic processes. In present study, methods of [³H]-HHT used for determined HHT. So far, it is best methods for research metabolism of HHT.

Methods

Animals: Adult DBA/2J male mice, 6 to 8 weeks old. All animals weighed approximately 25 g when used in experiments. Each member of which received identical dosed (i.p.) of HHT.

Tumor: P388 leukemic cells induced in mice by inoculation with L1210 or P388 leukemic cells (1.0×105).

After P388 induced in mice 5 days, 1 mg/kg of [³H]-HHT (100 μC/mg) was injected to mice. Volumes were 0.01 ml/g body weight. Mice were killed at 1, 3, 6 and 24 hours after injection of [³H]-HHT. The key data of HHT in metabolism process are determined counts per minute (CPM) of [³H]-HHT in organs. CPM is determined by liquid scintillation counting (LSC). The methods of LSC combined with radioactive of [³H]-HHT can be determined using a tracer of HHT in organs. After killed animals, organs and blood were obtained from P388 mice. Organs were weighted. 10 mg of organs were digested. 10 ml of scintillation solution (which containing 0.5% and 0.00% of POPOP) were added to digested solution and determined at LSC. Other processes are similar to the standard method.

Results and Discussion

CPM of [³H]-HHT of different organs is listed as the following table:

TABLE 15
Metabolism of HHT

CMP/mg organ

| Organs | 1 hour | 3 hours | 6 hours | 24 hours |
|---|---|---|---|---|
| Brain | 499 | 646 | 793 | 986 |
| Heart | 1403 | 1800 | 1193 | 893 |
| Liver | 4145 | 6718 | 4109 | 2175 |
| Spleen | 2828 | 2426 | 2198 | 2156 |
| Lung | 2100 | 2427 | 1788 | 1312 |
| Kidney | 6501 | 3121 | 3501 | 1065 |
| Intestine | 1638 | 3288 | 2475 | 1067 |
| Stomach | 1626 | 2321 | 3703 | 1823 |
| Bone | 500 | 560 | 505 | 480 |
| Contents of intestine | 3323 | 7215 | 6250 | 5009 |
| Contents of stomach | 6821 | 84375 | 100719 | 117914 |

TABLE 16
[³H]-HHT in blood

| | 1 h | 2 h | 4 h | 8 h | 16 h | 24 h |
|---|---|---|---|---|---|---|
| CPM | 1820 | 1650 | 1100 | 920 | 428 | 150 |

The data of Table 15 showed that after injected 1 hour, [³H]-HHT came to kidney, liver and spleen; after injected 3 hours, liver had highest CPM. The data of Table 16 indicated that CPM of [³H]-HHT was markedly decreasing in blood system. CPM of major organs is significantly decreasing after 2 hours. However, CPM of bone is slowly changed.

In conclusion, the data of metabolism of HHT showed the HHT could safely be used as a drug.

The preparation of drugs, which can be accomplished by the extraction method set forth above or any conventional methods for extracting the active principles from the plants. The novelty of the present invention resides in the mixture of the active principles in the specified proportions to produce drugs, and in the preparation of dosage units in pharmaceutically acceptable dosage form. The term “pharmaceutically acceptable dosage form” as used hereinabove includes any suitable vehicle for the administration of medications known in the pharmaceutical art, including, by way of example, capsules, tablets, syrups, elixirs, and solutions for parenteral injection with specified ranges of drugs concentration.

In addition, the present invention provides novel methods for treatment of cancer cells with produced safe pharmaceutical agent. It will thus be shown that there are provided compositions and methods which achieve the various objects of the invention and which are well adapted to meet the conditions of practical use.

As various possible embodiments might be made of the above invention, and as various changes might be made in the embodiments set forth above, it is to be understood that all matters herein described are to be interpreted as illustrative and not in a limiting sense.

1. A shirttail retainer for a shirt worn with pants,
a. wherein said shirt includes front and rear panels, each said panel having an upper portion, an opposite shirttail portion and a waist portion between said upper and shirttail portions, said waist portion having an outward facing surface, and
b. said pants include a waistband at the top with a radially inward facing surface adapted to contact said shirt waist portion when it is worn within said pants waistband, said shirttail retainer comprising:
i. a friction strip fixed to and extending around at least part of said inward facing surface of said pants waistband, and
ii. a plurality of passive friction elements circumferentially spaced around and fixed to said outward facing surface of said waist portion of said shirt, said friction elements on said shirt, when pressed into contact with said friction strip on said pants, as a result of said pants waistband being urged radially inward against said waist portion of said shirt, establishing a frictional force that resists movement of said shirt waist portion upward relative to said pants waistband.

2. A shirttail retainer according to claim 1, wherein said friction strip on said pants waistband is a continuous strip extending around at least the majority of the circumferential length.

3. A shirttail retainer according to claim 1, wherein each of said friction elements fixed to said shirt waist area comprises an elongated strip extending in a generally top-to-bottom direction.

4. A shirttail retainer according to claim 1, wherein said friction strip and said friction elements each comprise a thin generally flat layer of silicone rubber.

5. A shirt and microphone sling combination for releasably holding a microphone having an attachment clip, comprising,
a. a shirt having:
i. front and rear, right and left fabric panels, each panel having a top portion,
ii. a right shoulder between said top portions of said right front and rear panels and a left shoulder between said left front and rear panels, and
iii. a collar between said shoulders,
b. an epaulet affixed atop each of said shoulders,
c. first coupling means affixed atop at least one of said shoulders and beneath said epaulet thereon, and
d. a microphone sling formed as a strip having top and bottom parts, said top part having a second coupling means releasably coupled to said first coupling means beneath said epaulet with said strip hanging downward therefrom to lie adjacent and against said shirt's front panel, and said bottom part adapted to be engaged by said microphone clip for releasably attaching said microphone to said sling.

6. A combination according to claim 5, wherein said sling comprises a microphone coupling element on said sling bottom part.

7. A combination according to claim 6, wherein said microphone coupling element comprises a strap that extends laterally across said sling bottom part and having ends of said strap fixed to said bottom part, said strap being adapted to be engaged by said attachment clip.

8. A combination according to claim 5, wherein said first coupling means comprises a button and said second coupling means comprises a buttonhole in said top part of said sling.

9. A combination according to claim 5, comprising two of said first coupling means, each affixed atop one of said right and left shoulders beneath said epaulet thereon, said microphone sling being adapted to be releasably coupled to either of said first coupling means.

10. A method of releasably coupling a microphone to a person's shirt which has epaulets on both shoulders, a collar between said epaulets and a breast pocket below at least one of said epaulets, comprising:
a. providing a first coupling element on said shirt beneath each of said epaulets in a location that is essentially hidden from view,
b. providing a microphone sling having top and bottom parts, with a second coupling element fixed to said top part, and said bottom part being adaptable for attachment with a microphone,
c. said second coupling element being releasably couplable to either of said first coupling elements beneath said epaulets, such that said bottom part of said sling will hand downward and lie spaced apart from said collar and above said breast pocket, and said microphone being releasably attached to said bottom part of said microphone sling.

11. A pair of pants formed of fabric cloth, comprising:
a. an upper portion with a waistband,
b. left and right leg portions extending downward from said upper portion, each leg portion having a top part, a bottom cuff part and an intermediate part, each leg portion formed by front and rear panels which are joined along their side edges as inner and outer seams respectively, said seams extending from said cuff part upward to said upper portion of said pants, said inner seams of said two let portions being adjacent each other, and said outer seams being remote from each other, and
c. at least one internal cargo pocket formed by inner and outer panels, said panels having respective bottom and side edges joined together respectively as closed sides and a closed bottom of said cargo pocket, said cargo pocket being inside a pants leg with said outer panel of said cargo pocket adjacent said inner side wall of said pants leg, said panels having respectively top edges separate from each and other and defining a top opening of said cargo pocket, each of said top edges extending transversely across said outer seam of said leg portion and overlying and joined to parts of said front and rear panels adjacent said outer seam.

12. A pair of pants according to claim 1 1, further comprising, closure means for releasably joining said top edges of said cargo pocket to each other to close and open said cargo pocket.

13. A pair of pants according to claim 1 1, further comprising coupling means for releasably attaching said bottom of said cargo pocket to said outer seam.

14. A pair of pants according to claim 12, wherein said closure means comprises a zipper having releasably connectible strips joined to said top edges respectively of said cargo pocket.

15. A pair ofpants according to claim 13, wherein each of said leg portions defines an outer wall comprising said outer seam and longitudinal sections of said front and rear panels immediately adjacent said outer seam, said outer wall having an exposed outer surface and an opposite inner surface, said cargo pocket having its top opening extending transversely in said outer wall.

16. A pair of pants according to claim 15, further comprising an elongated stripe fixed to said outer wall of each of said leg portions along said outer seam and extending from said cuff part upward to said top portion of said pants.

17. A pair of pants according to claim 11, having said internal cargo pocket with bottom securely and removeably attached to one of said each leg portions further comprising a second similar internal cargo pocket similarly attached to the other of said leg portion.

18. A pair of pants according to claim 15, wherein each of said leg portions defines an outer wall comprising said outer seam and longitudinal sections of said front and rear panels immediately adjacent said outer seam, said outer wall having an exposed outer surface and an opposite inner surface, said cargo pocket having its top opening extending transversely in said outer wall.

19. A pair of pants according to claim 16, further comprising an elongated stripe fixed to said outer wall of each of said leg portions along said outer seam and extending from said cuff part upward to said top portion of said pants.

20. A pair of pants formed of fabric cloth, comprising:
a. an upper portion,
b. right and left leg portions extending downward from said upper portion, each of said leg portions comprising opposite outer and inner side walls with top and bottom parts of each side wall, and
c. an internal cargo pocket comprising inner and outer panels whose respective top edges define the opening of said pocket, said cargo pocket being inside a pants leg, with said outer panel of said cargo pocket adjacent said inner side wall of said pants leg, said outer side wall having a transverse opening situated between said top and bottom parts of said side wall, defining adjacent edges which are attached respectively to said top edges of said cargo pocket, whereby a user has access to said pocket via said opening in said outer wall and thence through said top opening of said pocket.

21. The combination of (a) a uniform consisting of a shirt having shoulders between front and rear panels and an epaulet atop each shoulder and a pair ofpants having an upper portion with a waistband and legs extending downward from said upper portion, each leg portion comprising outer and inner side walls with top and bottom parts of each side wall, (b) a shirttail retainer, (c) an internal cargo pocket in said pants, and (d) a microphone sling system on said shirt, where
aa. said shirttail retainer comprises:
i. a friction strip fixed to and extending around at least part of said inward facing surface of said pants waistband, and
ii. a plurality of friction elements circumferentially spaced around and fixed to said outward facing surface of said waist portion of said shirt, said friction strip and said friction elements each comprising a thin layer of silicone rubber, said friction elements on said shirt, when pressed into contact with said friction strip on said pants, as a result of said pants waistband being urged radially inward against said waist portion of said shirt, establishing a frictional force that resists movement of said shirt waist portion upward relative to said pants waistband,
bb. said internal cargo pocket comprises inner and outer panels whose respective top edges define the opening of said pocket, said cargo pocket being inside a pants leg, with said outer panel of said cargo pocket adjacent said inner side wall of said pants leg, said outer side wall having a transverse opening situated between said top and bottom parts of said side wall, defining adjacent edges which are attached respectively to said top edges of said cargo pocket, whereby a user has access to said pocket via said opening in said outer wall and thence through said top opening of said pocket, and
cc. said microphone sling system comprises:
i. an epaulet affixed atop each of said shoulders,
ii. a button affixed atop at least one of said shoulders and beneath said epaulet thereon, and
iii. a microphone sling formed as a strip having top and bottom parts, said top part having a button hole releasably attachable to said button beneath said epaulet with said strip hanging downward therefrom to lie adjacent and against said shirt's front panel, and said bottom part adapted to be engaged by said microphone clip for releasably attaching said microphone to said sling.
